(19) United States
(12) Patent Application Publication
(10) Pub. No.: US 2015/0004248 A1
(43) Pub. Date: Jan. 1, 2015

(54) METHOD AND DEVICE FOR GENERATING A NON-THERMAL PLASMA HAVING A PREDETERMINED OZONE CONCENTRATION

(71) Applicants: Max-Planck-Gesellschaft zur Förderung der Wissenschaften e.V, München (DE); University of California, Berkeley, Berkeley, CA (US)

(72) Inventors: Gregor Morfill, Munchen (DE); Tetsuji Shimizu, Garching (DE); Julia Zimmermann, Munchen (DE); Yang-Fang Li, Garching (DE); Yukinori Sakiyama, West Linn, OR (US); David Graves, Lafayette, CA (US)

(73) Assignees: University of California, Berkeley, Berkeley, CA (US); Max-Planck-Gesellschaft zur Föderung der Wissenschaften e.V., München (DE)

(21) Appl. No.: 14/358,811

(22) PCT Filed: Nov. 20, 2012
(86) PCT No.: PCT/EP2012/073139

§ 371 Date: Jun. 16, 2014

(30) Foreign Application Priority Data
Nov. 22, 2011 (DE) 102011119634.3

(51) Int. Cl. A61L 2/20; A61K 33/00; C01B 13/11

(52) U.S. Cl. CPC A61L 2/202; C01B 13/11; A61K 33/00; C01B 2201/10
USPC 424/613; 204/176; 422/29; 422/186.12

(57) ABSTRACT

A method for generating a non-thermal plasma having predetermined ozone concentration includes: providing an at least approximately closed volume as a reaction region; activating a plasma source and generating a non-thermal plasma in the reaction region. The plasma is held in the reaction region at least until a predetermined ozone concentration is reached or the ozone concentration falls below a predetermined upper limit for the ozone concentration.

The invention relates to a method for generating a non-thermal plasma according to the preamble of claim 1, and a device according to the preamble of claim 9.

Methods and devices for generating non-thermal plasmas are known. Non-thermal plasmas are preferably used in the medical field, in the domestic field, in the field of food, and/or in other fields for disinfection and/or sterilization, because in particular surface concentrations of bacteria, spores, viruses, fungi, and harmful biological material in general can be reduced with their aid. The plasmas are also used to inactivate annoying substances, for example, odor-forming or odoriferous molecules, allergens, prions or other unpleasant, health-endangering, otherwise impairing substances. Non-thermal plasmas can also be used to assist in wound healing, to combat skin inflammations, skin irritations, acne, for the relief of itching, in particular after insect bites, to relieve pain, as a deodorant, for physical hygiene, and in other manifold ways. Known methods and devices have the disadvantage that ozone arises during the generation of non-thermal plasmas in concentrations which impair or can endanger the health of the operator of a device or the operator who carries out the method, and/or a person who is to be treated using the non-thermal plasma.

The object of the invention is therefore to provide a method and a device, with the aid of which a non-thermal plasma can be generated, wherein at the same time an ozone concentration is reducible and/or settable.

The object is achieved in that a method having the features of claim 1 is provided.

The method for generating a non-thermal plasma with predetermined ozone concentration comprises the following steps: An at least approximately closed volume is provided as a reaction region. A plasma source is activated, and a non-thermal plasma is generated in the reaction region. The plasma is held in the reaction region at least until a predetermined ozone concentration is reached. The plasma chemistry changes chronologically, while the plasma is held in the reaction region. In this way, it is possible to set the ozone concentration with the aid of the dwell time of the plasma in the reaction region.

In this case, the formulation that a plasma having predetermined ozone concentration is generated also comprises the case that the ozone concentration falls below a predetermined upper limit for the ozone concentration, so that in particular a hazard for persons is no longer present.

Therefore, a method is alternatively preferable, in which a non-thermal plasma having reduced ozone concentration is generated, in that the plasma is held in the reaction region at least until the ozone concentration falls below a predetermined upper limit for the ozone concentration. In this case, the finally achieved end concentration of ozone is thus not predetermined, but rather it is ensured that the ozone concentration falls below a predetermined upper limit, whereby endangerment of persons can preferably be precluded.

Preferably, in the scope of the method, the ozone concentration to be reached is determined before the activation of the plasma source and the generation of the non-thermal plasma. In the same manner, in the alternative embodiment of the method, the upper limit for the ozone concentration is preferably determined for the activation of the plasma source and before the generation of the non-thermal plasma. The corresponding values for the ozone concentration or for the upper limit of the ozone concentration are accordingly established before the activation of the plasma source, so that they are predetermined in this regard. It is possible, to determine these values, to use predefined values, in particular limiting values, for example, a maximum workspace concentration for ozone or a permissible ozone limiting value, respectively. In particular, it is possible to establish such a value as the ozone concentration to be reached or as the upper limit for the ozone concentration. In a preferred embodiment of the method, a detection limit for ozone is determined as the upper limit for the ozone concentration.

A method is preferred in which the plasma source is kept activated until, after passing through a maximum of the ozone concentration in the reaction region, the predetermined ozone concentration is reached or the ozone concentration falls below the predetermined upper limit for the ozone concentration. The maximum of the ozone concentration has in this case a value which is greater than the predetermined ozone concentration or the predetermined upper limit for the ozone concentration. In the embodiment of the method described here, the ozone concentration always firstly passes through a maximum, before finally, with decreasing ozone concentration, the ozone concentration either reaches the predetermined ozone concentration or falls below the predetermined upper limit for the ozone concentration. In this case, the activated plasma source is used to intentionally and actively reduce the ozone concentration after passing through the maximum. In particular, ozone is broken down in this case by species generated by the plasma source. As will be shown hereafter, the plasma chemistry can be influenced particularly efficiently with the aid of the activated plasma source.

A method is preferred which is distinguished in that the plasma is transferred from the reaction region into an active region, after the ozone concentration has reached the predetermined ozone concentration or has fallen below the predetermined upper limit for the ozone concentration. In this case, the active region is set apart from the reaction region. In this way, it is ensured that the plasma first reaches the active region when the ozone concentration has reached the predetermined ozone concentration or has fallen below the predetermined upper limit, so that an ozone concentration which possibly impairs persons or is health-endangering is not present in the active region at any point in time. In particular, the setting of the ozone concentration in the plasma is chronologically and spatially separated from the actual plasma treatment, in order to prevent an undesired ozone concentration from penetrating into the active region.

It is possible in this case that the active region comprises a treatment region, in which the non-thermal plasma is used for disinfection and/or sterilization and/or assistance of wound healing. In this regard, by way of the spatial separation of active region and reaction region and by way of the chronological separation of the setting of the ozone concentration from the actual plasma treatment, for example, medical personnel and the patient being treated are not impaired by an excessively high ozone concentration.

An outlet device, which divides off the reaction region from the active region, is preferably moved from a first functional setting, in which the reaction region is separated from the active region, into a second functional setting, in which the reaction region has a fluid connection to the active region—preferably via the outlet device—to transfer the plasma from the reaction region into the active region. A closure or valve is particularly preferably used in this case as the outlet device, which blocks a fluid connection between the reaction region and the active region in the first functional setting, wherein it releases the fluid connection in the second functional setting.

A method is also preferred, which is distinguished in that a plasma is continuously expelled from the reaction region into the active region. This is preferably performed by means of a fluid stream.

In this case, the plasma source is preferably operated at a power which is adapted to a time, which the plasma requires to flow through a route from the plasma source up to an entry into the active region, such that upon entry of the plasma into the active region, the ozone concentration has reached the predetermined ozone concentration or has fallen below the predetermined upper limit for the ozone concentration. In this case, the reaction region and the active region have a permanent fluid connection to one another, wherein the plasma is driven by the fluid stream from the reaction region into the active region. To keep an ozone load in the active region low in this case, it is provided that the ozone concentration reaches the predetermined ozone concentration or falls below the predetermined upper limit for the ozone concentration before or at latest when the plasma reaches the active region. This is ensured in that the plasma source is operated at a power which ensures an effective, active reduction of the ozone concentration within the time which the plasma requires to flow through the route from the plasma source up to the entry into the active region. In particular, it can be provided that the plasma source is operated at a power which is sufficiently high that the reduction of the ozone concentration to the predetermined value or below the predetermined upper limit occurs quasi-instantaneously—in particular at a time scale relevant for the flow of the plasma.

The embodiment described here of the method is preferably carried out in a blower device, which is preferably implemented as a hairdryer, in particular a handheld hairdryer, or also a hand dryer, wherein the blower device has a plasma source. In this case, plasma expelled from the blower device in the direction toward a user or into the room air is to be prevented from having an ozone concentration which is impairing or health-endangering to the user. The power of the plasma source in the blower device is therefore preferably to be adapted to a time which the plasma requires from the plasma source until reaching an outlet out of the blower device, so that a corresponding reduction of the ozone concentration to a predetermined value or below the predetermined upper limit is ensured by this time.

Further advantageous embodiments result from the dependent claims.

The object is also achieved in that a device having the features of claim 9 is provided.

The device for generating a non-thermal plasma having predetermined ozone concentration comprises a plasma source and an at least approximately closed reaction region. The plasma source is arranged in this case relative to the reaction region so that a plasma can be generated therein. The device is distinguished by a setting means, with the aid of which a predetermined ozone concentration or a predetermined upper limit for an ozone concentration of the plasma to be generated is settable. The advantages already described in conjunction with the method result.

A device is preferred in which the setting means comprises a time switch element, by which an activation duration of the plasma source is predefinable. As will be shown hereafter, the ozone concentration of the plasma can thus be set or depressed below a predetermined upper limit very effectively. Alternatively or additionally, the setting means preferably comprises at least one variation element for variation of at least one parameter, wherein the at least one parameter is preferably selected from a group consisting of a pressure in the reaction region, a voltage of the plasma source, a frequency of the plasma source, an energy or power consumption of the plasma source, and a time which is available for a so-called afterglow of the plasma. The afterglow indicates that the plasma continues to exist for a certain time after the deactivation of the plasma source, wherein plasma chemistry still occurs in particular, i.e., chemical reactions run in the plasma. This afterglow is also referred to as afterglow. With the aid of a variation of at least one of the mentioned parameters, it is not only possible to set a predetermined ozone concentration or to depress the ozone concentration below a predetermined level, but rather it is also possible to set a stoichiometric ratio of ozone molecules and nitrogen oxide species.

A device is also preferred which has an active region set apart from the reaction region. The setting element has an outlet device, which is implemented so that the plasma can be held in the reaction region in a first functional setting of the outlet device. In a second functional setting of the outlet device, the plasma can be let out of the reaction region into the active region. The setting element is preferably implemented as the outlet device. It is possible with the aid of the outlet device to hold the plasma in the reaction region until the ozone concentration reaches a predetermined ozone concentration or falls below a predetermined upper limit for the ozone concentration, respectively. Only then is the plasma let out or transferred with the aid of the outlet device into the active region. In this way, it is ensured that an ozone concentration which could impair a person or be health-endangering is not present in the active region at any point in time. The active region is therefore spatially separated from the reaction region, so that an elevated ozone concentration occurring in the reaction region does not have a negative effect in the active region. At the same time, a chronological separation of the setting of the ozone concentration in the plasma and an actual plasma treatment is made possible. The active region is preferably a treatment region, in which a plasma treatment is carried out, in particular in the meaning of a disinfection and/or sterilization and/or an assistance of wound healing.

A device is preferred in which the outlet device is implemented as a closure or valve. The closure or the valve can be opened when the plasma is to be let out of the reaction region. During a setting of the ozone concentration, the plasma is held in the reaction region in that the closure or the valve remains closed. The outlet device is particularly preferably implemented so that, in the first functional setting, it blocks a fluid connection between the reaction region and the active region. Furthermore, it is preferably implemented so that it releases the fluid connection between the reaction region and the active region in the second functional setting. Accordingly, ozone cannot reach the active region from the reaction region when the outlet device is arranged in its first functional setting. In contrast, the plasma having reduced ozone concentration can be transferred from the reaction region into the active region when the outlet device is arranged in its second functional setting.

Finally, a device is preferred in which the outlet device comprises a fluid source and a channel. The fluid source has a fluid connection to the reaction region via the channel. The plasma can be let out of the reaction region into the active region continuously or in cycles, in that preferably a fluid stream is introduced through the channel into the reaction region, which displaces the plasma therefrom. A valve device is preferably provided, which is preferably arranged along the channel, and by which a fluid connection from the fluid source through the channel into the reaction region can be blocked in a first functional setting of the valve device and can be released in a second functional setting thereof.

It is then possible to expel the plasma in cycles from the reaction region into the active region, in that the valve device is opened after a predetermined reaction time, wherein the valve device remains closed while the ozone concentration is results.

Further advantageous embodiments result from the dependent claims.

The invention will be explained in greater detail hereafter on the basis of the drawing. In the figures:

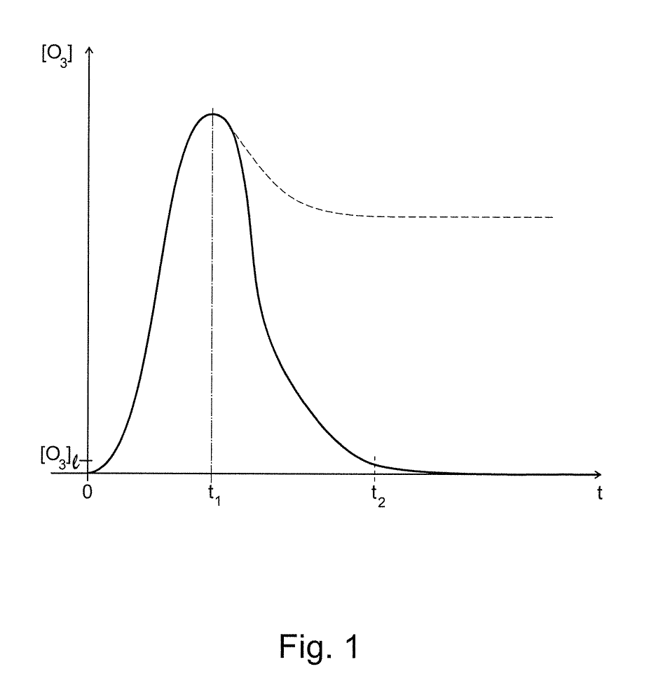

FIG. 1 shows a graph of an ozone concentration as a function of time;

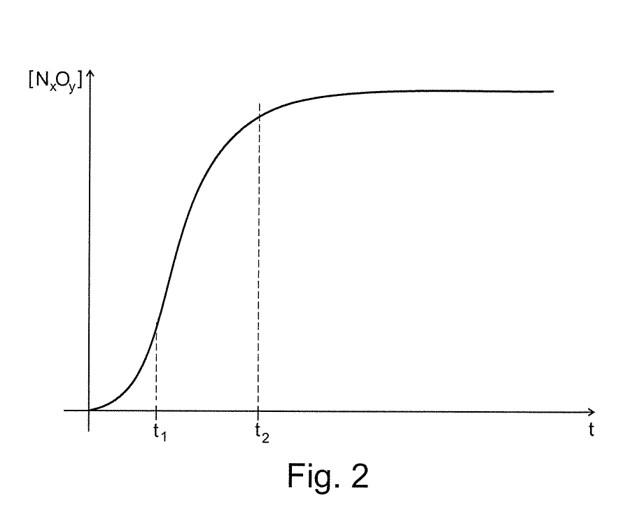

FIG. 2 shows a graph of a nitrogen oxide concentration as a function of time;

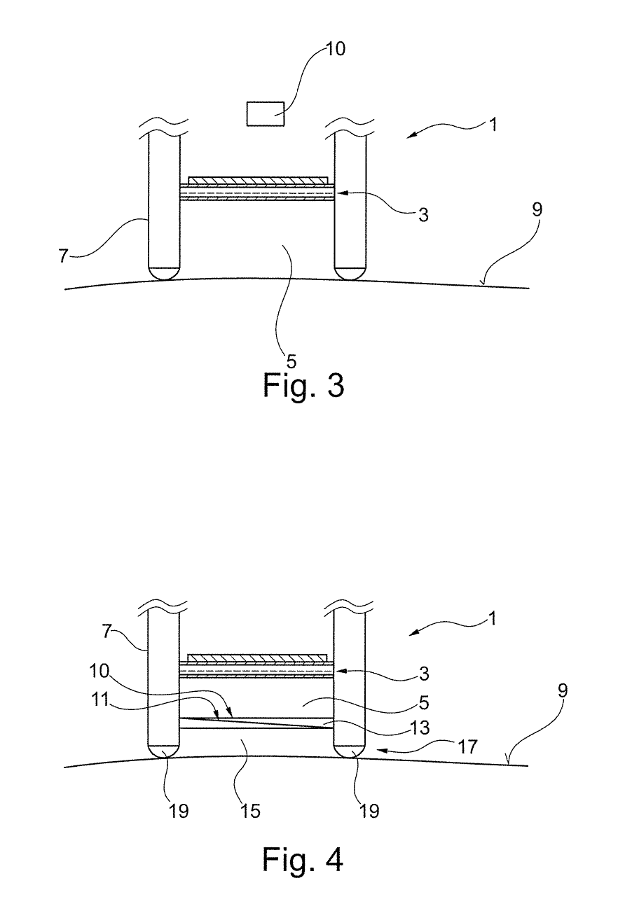

FIG. 3 shows a first exemplary embodiment of a device for generating a non-thermal plasma having predetermined ozone concentration;

FIG. 4 shows a second exemplary embodiment of the device;

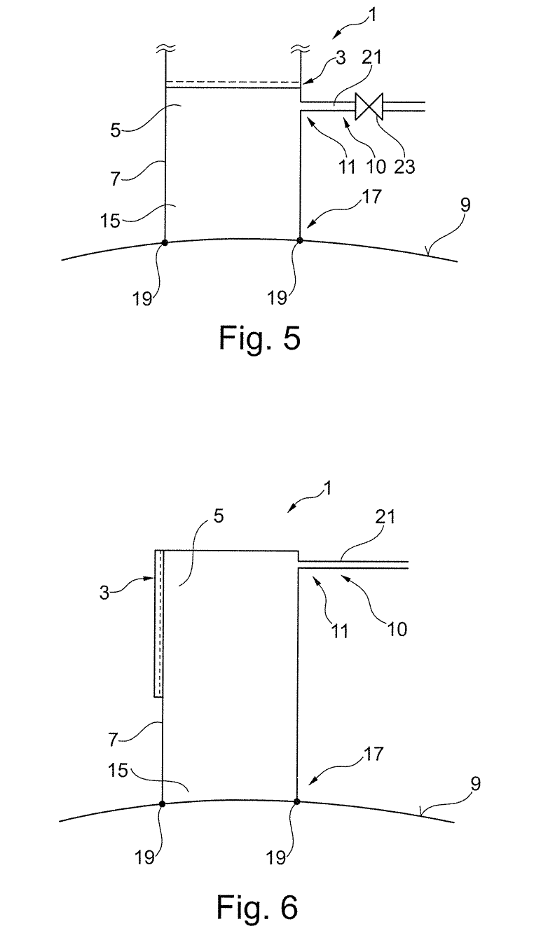

FIG. 5 shows a third exemplary embodiment of the device; and

FIG. 6 shows a fourth exemplary embodiment of the device.

FIG. 1 shows an ozone concentration [O₃] in an at least approximately closed volume as a function of time t.

The volume is at least approximately closed, which means that in any case a rate at which gas or plasma exits from the volume is negligibly small in comparison to a time constant, at which ozone is formed or decomposes again, respectively, in the reaction region. In other words, approximately closed means that a time constant, at which a relevant gas or plasma quantity is lost and/or exchanged from the reaction region, is large in comparison to a relevant time scale, at which a predetermined ozone concentration results or the ozone concentration falls below a predetermined upper limit of the ozone concentration.

The volume is particularly preferably closed so that the gas/plasma loss rate approaches zero or the time scale for the gas/plasma loss or exchange approaches infinity, respectively.

At a point in time t=0, a plasma source is activated. The solid line in FIG. 1 shows that the ozone concentration [O₃] first increases strongly and passes through a maximum at a point in time t₁. If the plasma source is deactivated at the point in time t₁, the ozone concentration [O₃] follows the curve shown by a dashed line.

If the plasma source remains activated, the ozone concentration follows [O₃] the curve shown by a solid line. It drops below a detection limit at a point in time t₂, which is identified here with [O₃]ₗ. This detection limit is preferably 10 ppm.

The plasma source is preferably operated at a surface power of 0.1 W/cm². The surface power is particularly preferably 0.2 W/cm². It has been shown that no ozone reduction occurs when the surface power of the plasma source is 0.05 W/cm².

It is essential that the plasma is generated in an at least approximately closed reaction region. If an open region is instead provided, the ozone concentration [O₃] does not rise to the maximum value shown in FIG. 1, but rather only to a significantly lesser maximum, nor does any effective reduction thereof occur.

As FIG. 1 shows, it is possible to set the ozone concentration [O₃], in that the plasma is held for at least a specific time in the reaction region. In this case, the plasma source can be deactivated at a specific point in time, for example, at the point in time t₁. However, it preferably remains activated until the ozone concentration reaches a predetermined ozone concentration or falls below a predetermined upper limit for the ozone concentration. For example, it is possible to keep the plasma source activated up to the point in time t₂, to actively depress the ozone concentration [O₃] below the detection limit [O₃]ₗ.

FIG. 2 shows a nitrogen oxide concentration [NₓO_{y}] as a function of time t. It has been shown that the nitrogen oxide concentration [NₓO_{y}] increases with the time t and finally passes into a saturation region, wherein an at least approximately constant value results. The points in time t₁ and t₂ are also shown on the time axis in FIG. 2, which correspond to the points in time t₁ and t₂ according to FIG. 1.

It is clear from a comparison of FIG. 1 and FIG. 2 that the kinetics of the ozone formation are determined by smaller time scales or greater rates than those of the nitrogen oxide formation. The ozone concentration [O₃] passes through its maximum at the point in time t₁, wherein the nitrogen oxide concentration [NₓO_{y}] is not yet very pronounced at this time.

Ozone is essentially formed by the following reactions:

O₂+e⁻→2O+e⁻ (1)

O+O₂+M→O₃+M (2)

In this case, e⁻ denotes a free electron from the plasma, and M denotes any type of third impact partner, which in particular absorbs oscillation energy from the O₃ molecule being created or its transition complex, so that its oscillation excitation drops below a dissociation threshold.

Nitrogen oxides are essentially formed by the following reactions:

N₂+e⁻→2N+e⁻ (3)

N+O→NO (4)

N+O₂→NO₂ (5)

N+O₃→NO+O₂ (6)

NO+O₃→NO₂+O₂ (7)

NO₂+O₃→NO₃+O₂ (8)

The speed constants of the ozone formation, in particular those of a speed-determining step, are greater than those for the formation of the nitrogen oxides, in particular speed constants which are relevant according to equations (3) to (5).

The ozone concentration [O₃] therefore initially rises. If the nitrogen oxides reach a relevant concentration, they react at relevant reaction speed with the ozone, so that it is finally broken down. This occurs essentially according to reaction equations (6) to (8).

It has thus been shown that at the point in time t₁, a nitrogen oxide concentration [NₓO_{y}] is reached, which has the result that the ozone concentration [O₃] does not increase further, passes through maximum, and finally decreases again. In this case, it is also clear according to above reaction equations (6) to (8) that the initially rising ozone concentration [O₃] itself contributes to nitrogen oxides increasingly being formed, which then react with ozone.

Toward longer times, the formation reactions of the nitrogen oxides dominate the plasma chemistry, so that the ozone concentration [O₃] finally sinks to an at least approximately constant value.

If the plasma source is deactivated at the point in time t₁, the ozone concentration [O₃] does sink under the level of the maximum according to FIG. 1, but it is not reduced under the detection limit [O₃]ₗ. It is similarly also possible in this case to passively set a predetermined ozone concentration in that the plasma is held for at least a predetermined time in the reaction region. It is also possible to generate a non-thermal plasma having reduced ozone concentration [O₃] in that the plasma is held in the reaction region until the ozone concentration falls below a predetermined upper limit for the ozone concentration [O₃]. However, this upper limit is restricted by the equilibrium concentration, which is achievable for long times.

If the plasma source is activated for a longer time beyond the point in time t₁, it is possible to actively reduce the ozone concentration [O₃] by influencing the plasma chemistry using the species generated by the activated plasma source after passing through the maximum shown in FIG. 1 to a predetermined value or below a predetermined upper limit, in particular to depress it under the detection limit [O₃]ₗ. For example, the plasma source can remain activated up to the point in time t₂, wherein the ozone concentration [O₃] then falls below the detection limit. It is thus possible to set a predetermined ozone concentration in that the plasma source is activated for a predetermined time, wherein the plasma is held in the reaction region at least for this time. It is also possible in this manner for the ozone concentration to fall below a predetermined upper limit for the ozone concentration. This upper limit can be a detection limit [O₃]ₗ in particular.

If one considers FIGS. 1 and 2, it is clear that a predetermined ratio of the ozone concentration [O₃] to a nitrogen oxide concentration [NₓO_{y}] can also be passively set via a dwell time of the plasma in the reaction region. Alternatively or additionally, this ratio can be set actively via an activation duration of the plasma source and an influence of the plasma chemistry connected thereto. The following is shown in this case: Since the nitrogen oxide concentration [NₓO_{y}] grows monotonously with time t, a setting of a predetermined ratio, which is actively performed with the aid of the plasma source, of the ozone concentration [O₃] to the nitrogen oxide concentration [NₓO_{y}] corresponds at the same time to a setting of a predetermined ozone concentration on the falling branch of the curve thereof shown in FIG. 1 after passing through the maximum. Therefore, an active setting of a predetermined ozone concentration also comprises an active setting of a predetermined ratio of the ozone concentration to the nitrogen oxide concentration.

It is possible to let the plasma out of the reaction region after the setting of a predetermined ozone concentration [O₃] or after the ozone concentration falls below a predetermined upper limit for the ozone concentration [O₃]. It is preferably provided that the plasma is conducted over into a treatment region or active region, respectively.

It is also possible that the plasma also remains in the reaction region after reaching the predetermined ozone concentration or after the ozone concentration falls below the predetermined upper limit thereof in the reaction region. In particular, it is possible to generate the plasma in a closed volume, which is closed by a cover, a film, a plastic skin, or a similar element, to set a predetermined ozone concentration [O₃] or to fall below an upper limit thereof, respectively, and finally to let the plasma act on the closed volume, in particular on a surface of walls of the closed volume. In this manner it is possible, for example, to treat containers of packaged foods, for example, yogurt in a yogurt cup, or other packaged objects with the aid of the plasma, preferably to sterilize them. It is also possible to generate the plasma within a volume enclosed by an adhesive bandage or a dressing or a similar element, and correspondingly to set a predetermined ozone concentration or to depress this below a predetermined upper limit, respectively. An electrode of the plasma source is preferably applied from the outside to a wall, particularly preferably a cover, a film, a plastic skin, or a similar element, an adhesive bandage, or a dressing. It is then possible to generate a plasma in the volume enclosed thereby. Finally, it is also possible to set a predetermined nitrogen/oxygen stoichiometry of the plasma in the closed volume, in particular a stoichiometric ratio of the ozone concentration [O₃] to the nitrogen oxide concentration [NₓO_{y}].

In addition to an activation duration of the plasma source, further parameters are relevant for setting the ozone concentration or the nitrogen/oxygen stoichiometry of the plasma, respectively. These parameters comprise a pressure in the reaction region, a voltage at which the plasma source is operated, a frequency which is applied to the electrodes of the plasma source, an energy or power consumption of the plasma source, and a time which is available for an afterglow, i.e., a so-called afterglow of the plasma.

As already stated, a surface power of the plasma source is at least relevant insofar as reduction of the ozone concentration [O₃] no longer occurs below a specific surface power level. This is presumably because at least nitrogen oxides are no longer generated, which have a sufficient oscillation excitation to react according to reaction equations (6) to (8) with ozone, below a specific limiting value, presumably below a surface power of approximately 0.1 W/cm².

The non-thermal plasma is preferably used for the disinfection and/or sterilization in particular of surfaces, in particular also of skin and/or to assist in wound healing.

FIG. 3 shows a device for carrying out the method for generating a non-thermal plasma having predetermined ozone concentration. The device 1 comprises a plasma source 3 and an at least approximately closed reaction region 5. At least one wall 7 is provided, which encloses the reaction region 5. The illustrated device 1 is placed on a surface 9, which is to be disinfected, sterilized, or treated, respectively, with the aid of the non-thermal plasma. This can be skin in this case, in particular human skin. For example, wound healing can be assisted. It is also possible to treat allergies, itching, in particular as a result of insect bites, acne, or skin irritation, or to use the device 1 as a deodorant device, which inactivates odor-forming bacteria or reduces the concentration thereof, respectively, and/or destroys odor-relevant molecules, in particular odoriferous molecules or molecules which participate in odor formation.

The plasma source 3 is arranged relative to the reaction region 5 so that a plasma can be generated therein. It is fundamentally possible to imelement the plasma source 3 arbitrarily. However, it is preferably implemented as a surface micro discharge source (SMD), for example, as described in WO 2010/094304 A1, or according to the principle of the self-sterilizing surface (SSS), as described, for example, in EP 10005236.4, PCT/EP2011/002506, and in WO 2011/110343 A1. It is also possible that the plasma source 3 is implemented according to the principle of dielectric barrier discharge (DBD). The cited applications and documents are incorporated here by reference.

To carry out the method with the aid of the device 1, it is placed on the surface 9 to be treated. In this way, the reaction region 5 is implemented as the at least approximately closed volume. The plasma source 3 is then activated, and a non-thermal plasma is generated in the reaction region 5.

This plasma is held in the reaction region until a predetermined ozone concentration is reached or the ozone concentration falls below a predetermined upper limit for the ozone concentration. Only then is the device 1 removed from the surface 9. The gases or the plasma escaping from the reaction region 5 are/is then no longer harmful for a person who operates the device 1 or is treated thereby.

The plasma source 3 is preferably kept activated until a predetermined ozone concentration is reached or the ozone concentration falls below a predetermined upper limit for the ozone concentration. It is also possible to deactivate the plasma source 3 beforehand and then still wait a specific time until the desired ozone concentration has resulted or it has fallen below the desired upper limit, respectively.

In the device according to FIG. 3, the plasma acts on the surface 9 during an entire reaction time from the activation of the plasma source 3 up to a removal of the device 1 and/or a recombination of the plasma and therefore its decomposition.

The device 1 preferably comprises a setting means 10, with the aid of which the predetermined ozone concentration is settable. In the exemplary embodiment shown, the setting means 10 cooperates with the plasma source 3 and is preferably implemented as a time switch element, by which a predetermined activation duration of the plasma source 3 is predefinable. It is possible in this case that a predetermined ozone concentration or a predetermined upper limit for the ozone concentration can be input by a user of the device 1, wherein the setting means 10 itself converts this into a predetermined activation duration of the plasma source 3. It is also possible that a user of the device 1 directly predefines the activation duration of the plasma source 3.

In another exemplary embodiment, the setting means 10 additionally or alternatively comprises a variation means for variation of at least one parameter, wherein the parameter is preferably selected from a group consisting of a pressure in the reaction region 5, a voltage applied to the electrodes of the plasma source 3, a frequency of the voltage, which is preferably applied as AC voltage, an energy consumption or power consumption of the plasma source 3, and a time which is available for an afterglow of the plasma, a so-called afterglow. For example, it is possible to quench the plasma by suitable measures, i.e., to intentionally supply it to decomposition, whereby a time for the afterglow can be set.

In one exemplary embodiment of the device 1, it is possible to have an electrode of the plasma source 3 act on a cover, a film, a plastic skin, an adhesive bandage, a dressing, or a similar element which encloses a volume, to generate a plasma in the enclosed volume. It is then possible to generate a plasma having predetermined ozone concentration or ozone concentration reduced below a predetermined upper limit in the volume with the aid of the method or the setting means of the device 1, respectively. The reaction region 5 is then arranged in the closed volume. In this exemplary embodiment, the plasma remains in the closed volume also after setting of the predetermined or reduced ozone concentration. This can be in this case, for example, a container, preferably a food container, for example, a yogurt cup. It is also possible that the closed volume is implemented under an adhesive bandage, a dressing, or a similar element.

FIG. 4 shows a second exemplary embodiment of a device 1 according to the invention. Identical and functionally-identical elements are provided with identical reference signs, so that reference is made in this regard to the description of FIG. 3. The device 1 comprises a setting means 10 implemented as an outlet device 11 here, wherein the outlet device 11 has at least two functional settings. In a first functional setting thereof, the plasma can be held in the reaction region 5. In a second functional setting, it can be let out of the reaction region 5. The outlet device 11 is implemented as a closure 13 in the illustrated exemplary embodiment.

In its first functional setting, the closure 13 is closed, so that the plasma is held in the reaction region 5 enclosed by the wall 7 and the closure 13. If a predetermined ozone concentration is reached, the closure 13 is opened, wherein the plasma can diffuse in this second, open functional setting of the closure 13 out of the reaction region 5 into an active reBion 15. It is therefore shown that the closure 13 divides the reaction region 5 from the active region 15, so that the active region 15 is set off or spatially separated, respectively, from the reaction region. A fluid connection is blocked in this case between the reaction region 5 and the active region 15 in the first functional setting of the closure 13, while it is released in its second functional setting.

A distance between the closure 13 and the service 9 is preferably small in comparison to a distance of the closure 13 from the plasma source 3. In particular, the distance between the surface 9 and the closure 13, namely a height of the active region 15, is preferably small in comparison to a height of the reaction region 5—measured in the same direction. In this case, the diffusion length which the plasma must overcome in the event of opening of the closure 13 is small and it reaches the surface 9 rapidly.

A volume of the active region 15 is particularly preferably small in comparison to a volume of the reaction region 5.

The plasma is preferably let out of the reaction region 5 into the active region 15 in cycles. Plasma is generated at predetermined ozone concentration in each cycle in this case.

The closure 13 in the exemplary embodiment shown in FIG. 4 preferably opens when the ozone concentration reaches or falls below a predetermined ozone concentration, wherein the closure lets the plasma out of the reaction region 5 toward the surface 9. After a preferably predetermined treatment duration, the closure 13 closes again, and plasma having predetermined ozone concentration is again generated. This is continued until a desired treatment duration or a predetermined number of cycles is reached.

The plasma source 3 is preferably deactivated between the individual cycles. It is particularly preferably deactivated in an outlet face of the plasma.

In the exemplary embodiment shown in FIG. 4, the outlet phase is the time in which the closure 13 is open. If the plasma source is deactivated in this time, newly formed ozone cannot reach the surface 9.

It is shown that in the device 1 according to FIG. 4, the surface 9 is not stressed at any point in time with an ozone concentration which is higher than the predetermined ozone concentration or its upper limit, respectively.

The device 1 preferably comprises a distal wall region 17, which is provided for preferably sealed contact on a region to be treated, on the surface 9 here. It preferably comprises a soft and/or elastic material 19. This ensures a sealed terminus of the distal wall region 17 with the surface 9, on the one hand, a contact of the device 1 with the surface 9 is much more pleasant for a person to be treated, in particular in the case in which the surface 9 has skin, if a soft and/or elastic material is provided in the distal wall region 17, on the other hand.

FIG. 5 shows a third exemplary embodiment of the device 1. Identical and functionally-identical elements are provided with the same reference signs, so that in this regard reference is made to the preceding description. The reaction region 5 is only approximately closed here. It is arranged in the region of the plasma source 3 and is spaced apart from the surface 9 by the wall 7, which is implemented here having suitable length, so that a time scale determined by a diffusion of the plasma is sufficiently long so that the plasma is held in the reaction region 5 until a predetermined ozone concentration is reached.

The setting means 10, which is implemented as the outlet device 11, comprises here a channel 21, which has a fluid connection to the reaction region 5. The outlet device 11 preferably also comprises a valve device 23, by which a fluid connection through the channel 21 into the reaction region 5 can be blocked in the first functional setting of the outlet device 11 and can be released in its second functional setting.

The channel 21 preferably has a fluid connection at its end facing away from the reaction region 5 to a fluid source (not shown), for example, a gas bottle, a ventilator, or a blower, in general a means for generating a fluid stream.

As long as the valve device 23 is blocked, the plasma remains essentially in the reaction region 5. If the valve device 23 is opened, the plasma is let out of the reaction region 5, in that it is quasi-blown out or displaced by the fluid stream entering through the channel 21 into the reaction region 5, respectively. It thus reaches the active region 15.

With the aid of the valve device 23, the plasma is preferably let out of the reaction region 5 in cycles. Plasma having predetermined ozone concentration is preferably generated in each cycle in this case.

The plasma source is preferably also deactivated here between the individual cycles, in particular in an outlet phase of the plasma in which the valve device 23 is open, so that no newly formed ozone reaches the surface 9.

In another exemplary embodiment, it is possible to leave out the valve device 23 or only to set a preferably predetermined flow rate through the channel 21, particularly preferably to regulate it, by way thereof. The plasma is then continuously expelled from the reaction region, in particular with the aid of the fluid stream entering the reaction region 5 through the channel 21.

The flow speed is then preferably selected so that the plasma remains for a sufficiently long time in the reaction region 5 that a predetermined or reduced ozone concentration is reached. Alternatively, it is possible to adapt a power of the plasma source 3 to a time which the plasma requires to flow through a route from the plasma source 3 up to an entry into the active region 15. The power of the plasma source is selected so that, at latest upon entry of the plasma into the active region 15, the predetermined ozone concentration is reached or the ozone concentration falls below the predetermined upper limit for the ozone concentration, therefore a reduced ozone concentration is reached. It is also possible to adapt or tailor, respectively, both the flow speed and also the power of the plasma source 3 to one another, to achieve the desired result.

Layering particularly preferably results between the reaction region 5 and the active region 15, wherein the ozone concentration decreases along this layering until it reaches its predetermined value or falls below a predetermined upper limit in the active region 15.

FIG. 6 shows a fourth exemplary embodiment of the device 1. Identical and functionally-identical elements are provided with identical reference signs, so that reference is made in this regard to the preceding description.

As in the above-mentioned exemplary embodiment, a continuous fluid stream is also generated here through the channel 21 into the reaction region 5. However, in contrast to beforehand, the plasma source 3 is not arranged essentially opposite to the surface 9 to be treated here, but rather it extends along the wall 7 in the direction toward the surface 9. It is possible in this case that it extends directly or nearly directly up to the surface 9. However, the end of the plasma source 3 facing toward the surface 9 is preferably spaced apart from the surface 9.

The reaction region 5 extends along the plasma source 3. The dwell time of the plasma in the region of the plasma source 3 and therefore here in the reaction region 5 is then determined by the rate at which the plasma moves along the plasma source 3 toward the surface 9.

The ozone concentration of the plasma which reaches the surface 9 is therefore finally predefinable by the flow rate.

The outlet device 11 can preferably have a valve device (not shown), through which the flow rate through the channel 21 and therefore also the flow rate of the plasma to the surface 9 is settable. The corresponding flow rates can particularly preferably be controlled and/or regulated.

The distance between the surface 9 and the end of the plasma source 3 facing it determines, jointly with the flow rate of the fluid stream through the channel 21, the time which is available for an afterglow of the plasma. Namely, this corresponds to the time which the plasma needs to reach the surface 9 from the end of the plasma source 3 facing toward the surface 9. The time for the afterglow can therefore be set in that the flow rate through the channel 21 is varied. However, at the same time this also changes the dwell time of the plasma in the region of the plasma source 3. In a preferred exemplary embodiment, it is provided that the distance of the end of the plasma source 3 facing toward the surface 9 to the surface 9 is variable. A variation means is provided for this purpose, which can comprise an internal thread and an external thread, for example, which mesh with one another, so that a region of the wall 7 facing toward the surface 9 is displaceable relative to a main body of the device 1—viewed in the longitudinal direction. It is also possible to provide a click mechanism, guide grooves, and/or a fastening mechanism for various spacer parts. With the aid of the variation means for the distance, it is possible to vary the time for the afterglow independently of the dwell time of the plasma in the region of the plasma source 3. The time which is available for the afterglow of the plasma also influences its ozone concentration or a stoichiometric ratio between the ozone concentration and the nitrogen oxide concentration thereof, respectively, because chemical reactions still run during the afterglow, which have influence thereon.

In particular in the exemplary embodiment according to FIG. 6, it is possible that the plasma source 3 remains continuously activated. Since the ozone concentration of the plasma which reaches the active region 15 is determined in particular by the flow rate at which the plasma passes through the reaction region 5, an excessively high ozone concentration cannot reach the surface 9 or the active region 15, respectively, even if the plasma source 3 is permanently activated.

Alternatively or additionally, it is also possible to adapt the power of the plasma source 3, to cause the ozone concentration of the plasma to fall below a predetermined upper limit or have a predetermined value upon the entry of the plasma into the active region 15. In particular, it is possible to select the power of the plasma source 3 and the flow rate suitably or to tailor them to one another. The power of the plasma source 3, the flow rate, and the distance of the end of the plasma source 3 facing toward the surface 9 from the surface 9 are particularly preferably tailored to one another and adapted so that the desired result is achieved.

Overall, it has been shown that it is possible with the aid of the device and the method to generate a non-thermal plasma having predetermined ozone concentration and thus particularly preferably to protect the user of the device 1 and/or a person treated using the plasma from an excessively high ozone concentration.

1. A method for generating a non-thermal plasma having predetermined ozone concentration, the method comprising:
providing an at least approximately closed volume as a reaction region; and
activating a plasma source and generating a non-thermal plasma in the reaction region,
in which the plasma is held in the reaction region at least until a predetermined ozone concentration is reached or the ozone concentration falls below a predetermined upper limit for the ozone concentration.

2. The method of claim 1, wherein the plasma source is kept activated until, after passing through a maximum of the ozone concentration in the reaction region, the predetermined ozone concentration is reached or the ozone concentration falls below the predetermined upper limit for the ozone concentration, wherein the maximum of the ozone concentration has a value which is greater than the predetermined ozone concentration or the predetermined upper limit for the ozone concentration.

3. The method of claim 1, wherein the plasma source is kept activated until a predetermined ratio of the ozone concentration to a nitrogen oxide concentration is reached.

4. The method of claim 1, wherein the non-thermal plasma is used for disinfection and/or sterilization and/or assistance of wound healing.

5. The method of claim 1, wherein the plasma is transferred from the reaction region into an active region set apart from the reaction region after the predetermined ozone concentration is reached or the ozone concentration falls below the predetermined upper limit for the ozone concentration, wherein an outlet device, which divides the reaction region from the active region, is preferably moved for this purpose from a first functional position, in which the reaction region is separated from the active region, into a second functional position, in which the reaction region has a fluid connection to the active region.

6. The method of claim 5, wherein the plasma is transferred in cycles from the reaction region into the active region, wherein plasma having predetermined ozone concentration is generated in each cycle.

7. The method of claim 6, wherein the plasma source is deactivated between the individual cycles, in particular in an outlet phase of the plasma.

8. The method of claim 1, wherein the plasma is continuously expelled out of the reaction region into the active region, preferably by means of a fluid stream, wherein preferably the plasma source is operated at a power, which is adapted to a time which the plasma needs to flow through a route from the plasma source up to an entry into the active region, so that upon entry of the plasma into the active region, the predetermined ozone concentration is reached or the ozone concentration has fallen below the predetermined upper limit for the ozone concentration.

9. A device for generating a non-thermal plasma having predetermined ozone concentration, the device comprising:
a plasma source;
an at least approximately closed reaction region, and
a setting means, by which a predetermined ozone concentration or a predetermined upper limit for an ozone concentration of the plasma to be generated is settable,
wherein the plasma source is arranged in relation to the reaction region so that a plasma can be generated therein.

10. The device of claim 9, wherein the setting means comprises a time switch element, by which an activation duration of the plasma source is predefinable, and/or at least one means for variation of at least one parameter, wherein the at least one parameter is preferably selected from a group consisting of a pressure in the reaction region, a voltage, a frequency, and energy consumption or power consumption of the plasma source, and a time which is available for an afterglow of the plasma.

11. The device of claim 9, wherein:
the device has an active region, which is set apart from the reaction region, wherein
the setting element comprises an outlet device, which is implemented so that the plasma can be held in the reaction region in a first functional setting of the outlet device, and
can be let out of the reaction region into the active region in a second functional position.

12. The device of claim 9, wherein the outlet device is implemented as a closure or as a valve, wherein preferably the outlet device blocks a fluid connection between the reaction region and the active region in the first functional setting, wherein it releases the fluid connection in the second functional setting.

13. The device of claim 9, wherein the outlet device comprises a fluid source, a channel, which connects the fluid source to the reaction region, and preferably a valve device, which is preferably arranged along the channel, and by which a fluid connection from the fluid source through the channel into the reaction region can be blocked in a first functional position of the valve device and can be released in a second functional position.

14. The device of claim 9, further comprising at least one distal wall region, which is provided for preferably sealed contact on a region to be treated, wherein the distal wall region comprises a soft and/or elastic material.
